(19) United States
(12) Patent Application Publication
(10) Pub. No.: US 2025/0134585 A1
(43) Pub. Date: May 1, 2025

(54) SINGLE SHAFT LEAFLET MODIFICATION DEVICE

(71) Applicant: BOSTON SCIENTIFIC SCIMED, INC., MAPLE GROVE, MN (US)

(72) Inventors: Lauren Koon, Mississauga (CA); Gareth Davies, Toronto (CA); Prateek Mathur, Toronto (CA)

(73) Assignee: BOSTON SCIENTIFIC SCIMED, INC., MAPLE GROVE, MN (US)

(21) Appl. No.: 18/933,520

(22) Filed: Oct. 31, 2024

Related U.S. Application Data
(60) Provisional application No. 63/594,594, filed on Oct. 31, 2023.

(51) Int. Cl. A61B 18/14; A61B 18/00

(52) U.S. Cl. CPC A61B 18/1492; A61B 18/1445; A61B 2018/00077; A61B 2018/00083; A61B 2018/00136; A61B 2018/00184; A61B 2018/00369; A61B 2018/00595; A61B 2018/1422

(57) ABSTRACT

A medical assembly includes a guide catheter with a lumen extending therethrough and an electrocautery member slidingly disposed within the lumen. The electrocautery member has an elongate shaft with a proximal shaft segment and a distal shaft segment adapted to be movable between a puncture position and a laceration position. In some cases, a hinge segment is disposed between the proximal shaft segment and the distal shaft segment and includes a leading edge adapted to form a puncture within a cardiac valve leaflet when the distal shaft segment is in its puncture position and a trailing edge adapted to lacerate the cardiac valve leaflet when the distal shaft segment is in its laceration position. In some cases, the electrocautery member includes electrically isolated puncture and lacerating electrodes.

### CROSS-REFERENCE TO RELATED APPLICATIONS

This application claims the benefit of priority of U.S. Provisional Application No. 63/594,594 filed Oct. 31, 2023, the entire disclosure of which is hereby incorporated by reference.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, for example, intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating cardiac valve leaflets. An example may be found in a medical assembly. The medical assembly includes a guide catheter having a distal end and a lumen extending proximally from the distal end, and an electrocautery member that is adapted to be slidingly disposed within the lumen in order to puncture and lacerate a cardiac valve leaflet. The electrocautery member includes a shaft, a puncture electrode disposed relative to the shaft and a lacerating electrode disposed relative to the shaft. The shaft includes a proximal shaft segment, a distal shaft segment including a distal end region, and a hinge segment that is disposed between the proximal shaft segment and the distal shaft segment. The electrocautery member is adapted to lacerate the cardiac valve leaflet without excising part of the cardiac valve leaflet.

Alternatively or additionally, the puncture electrode may be disposed within the distal end region of the distal shaft segment.

Alternatively or additionally, the lacerating electrode may include an electrically exposed portion of the hinge segment.

Alternatively or additionally, the puncture electrode may include a leading edge of the hinge segment.

Alternatively or additionally, the lacerating electrode may include a trailing edge of the hinge segment.

Another example may be found in a medical assembly. The medical assembly includes a guide catheter including a distal end and a lumen extending proximally from the distal end, and an electrocautery member adapted to be slidingly disposed within the lumen in order to puncture and lacerate a cardiac valve leaflet. The electrocautery member has a single shaft including a proximal shaft segment, a distal shaft segment, and a hinge segment disposed between the proximal shaft segment and the distal shaft segment. The hinge segment includes a leading edge adapted to form a puncture within the cardiac valve leaflet and a trailing edge adapted to lacerate the cardiac valve leaflet. The electrocautery member is adapted to lacerate the cardiac valve leaflet without excising part of the cardiac valve leaflet.

Alternatively or additionally, the single shaft may include a conductive core extending through the proximal shaft segment, the hinge segment and the distal shaft segment, and an insulative polymeric sheath extending over the conductive core, the insulative polymeric sheath electrically insulating the proximal shaft segment and the distal shaft segment.

Alternatively or additionally, the leading edge of the hinge segment may not be covered via the insulative polymeric sheath.

Alternatively or additionally, the trailing edge of the hinge segment may not be covered via the insulative polymeric sheath.

Alternatively or additionally, the conductive core may include nitinol.

Alternatively or additionally, the electrocautery member may be adapted to be disposed relative to the guide catheter with the distal shaft segment extending proximally within the lumen in order to position the leading edge of the hinge segment to form the puncture within the cardiac valve leaflet.

Alternatively or additionally, the electrocautery member may be adapted to be disposed relative to the guide catheter with the distal shaft segment extending distally of the lumen in order to position the trailing edge of the hinge segment to lacerate the cardiac valve leaflet.

Alternatively or additionally, the proximal shaft segment and the distal shaft segment may form an acute angle at the hinge segment.

Alternatively or additionally, the distal shaft segment may include an atraumatic tip formed at a distal region of the distal shaft segment.

Another example may be found in a medical assembly. The medical assembly includes a guide catheter including a distal end and a lumen extending proximally from the distal end, and an electrocautery member adapted to be slidingly disposed within the lumen and to puncture and lacerate a cardiac valve leaflet without excising the cardiac valve leaflet. The electrocautery member includes a single shaft including a conductive core and an insulative polymeric sheath extending over the conductive core. The single shaft includes a proximal shaft segment in which the insulative polymeric sheath covers the conductive core, a distal shaft segment in which the insulative polymeric sheath covers the conductive core, and a hinge segment disposed between the proximal shaft segment and the distal shaft segment, the insulative polymeric sheath not covering at least part of the hinge segment.

Alternatively or additionally, the hinge segment may include a leading edge adapted to form a puncture within the cardiac valve leaflet and a trailing edge adapted to lacerate the cardiac valve leaflet.

Alternatively or additionally, the conductive core may include nitinol.

Alternatively or additionally, the electrocautery member may be adapted to be disposed relative to the guide catheter with the distal shaft segment extending proximally within the lumen in order to position the leading edge of the hinge segment to form the puncture within the cardiac valve leaflet.

Alternatively or additionally, the electrocautery member may be adapted to be disposed relative to the guide catheter with the distal shaft segment extending distally of the lumen in order to position the trailing edge of the hinge segment to lacerate the cardiac valve leaflet.

Alternatively or additionally, the proximal shaft segment and the distal shaft segment may form an acute angle at the hinge segment.

Alternatively or additionally, the distal shaft segment may include a distal tip that doubles over on itself.

Another example may be found in a medical assembly. The medical assembly includes a guide catheter including a distal end and a lumen extending proximally from the distal end, and an electrocautery member adapted to be slidingly disposed within the lumen and to puncture and lacerate a cardiac valve leaflet without excising the cardiac valve leaflet. The electrocautery member has a single shaft including a proximal shaft segment adapted to be slidingly disposed within the lumen, a distal shaft segment adapted to be movable between a puncture position in which the distal shaft segment extends proximally into the lumen and a laceration position in which the distal shaft segment extends distally away from the guide catheter, and a hinge segment disposed between the proximal shaft segment and the distal shaft segment. The hinge segment includes a leading edge adapted to form a puncture within the cardiac valve leaflet when the distal shaft segment is in its puncture position and a trailing edge adapted to lacerate the cardiac valve leaflet when the distal shaft segment is in its laceration position.

Alternatively or additionally, the single shaft may include a conductive core extending through the proximal shaft segment, the hinge segment and the distal shaft segment, and an insulative polymeric sheath extending over the conductive core, the insulative polymeric sheath electrically insulating the proximal shaft segment and the distal shaft segment.

Alternatively or additionally, the insulative polymeric sheath may not cover the leading edge or the trailing edge of the hinge segment.

Alternatively or additionally, the distal shaft segment may include an atraumatic tip formed at a distal region of the distal shaft segment.

Another example may be found in a medical assembly. The medical assembly includes a guide catheter including a distal end and a lumen extending proximally from the distal end, and an electrocautery member that is adapted to be slidingly disposed within the lumen and to puncture and lacerate a cardiac valve leaflet without excising the cardiac valve leaflet. The electrocautery member includes a shaft having a proximal shaft segment and a distal shaft segment. A puncture electrode is disposed within the distal end region. A lacerating electrode is disposed between the proximal shaft segment and the distal shaft segment.

Alternatively or additionally, the shaft may include a first conductive member that is electrically coupled with the puncture electrode and a second conductive member that is electrically coupled with the lacerating electrode.

Alternatively or additionally, the first conductive member and the second conductive member may be electrically isolated from each other via an electrically insulating material encasing the first conductive member and the second conductive member.

Alternatively or additionally, the puncture electrode may include a portion of the first conductive member exposed by a portion of the electrically insulating material being removed.

Alternatively or additionally, the puncture electrode may include a terminus of the first conductive member.

Alternatively or additionally, the lacerating electrode may include a portion of the second conductive member exposed by a portion of the electrically insulating material being removed.

Alternatively or additionally, the lacerating electrode may include a terminus of the second conductive member.

Alternatively or additionally, the first conductive member and the second conductive member may each include nitinol.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative medical assembly for lacerating a cardiac valve leaflet;

FIG. 2 is a schematic view of an illustrative electrocautery member forming part of the illustrative medical assembly of FIG. 1;

FIG. 3 is a cross-sectional view taken along the line 3-3 of FIG. 2;

FIG. 4 is a cross-sectional view taken along the line 4-4 of FIG. 2;

FIG. 5 is a schematic view of an illustrative electrocautery member;

FIG. 6 is a cross-sectional view taken along the line 6-6 of FIG. 5;

FIG. 7 is a schematic view of an illustrative electrocautery member;

FIG. 8 is a schematic view of an illustrative electrocautery member;

FIGS. 9 through 14 illustrate use of the illustrative medical assembly of FIG. 1 in lacerating a cardiac valve leaflet;

FIG. 15 is a schematic view of an illustrative electrocautery member;

FIG. 16 is a schematic view of an illustrative electrocautery member;

FIG. 17 is a schematic view of an illustrative electrocautery member;

FIG. 17A is a cross-sectional view taken along the line 17A-17A of FIG. 17;

FIG. 17B is a cross-sectional view taken along the line 17B-17B of FIG. 17; and

FIGS. 18 through 21 illustrate use of the illustrative electrocautery member of FIG. 17 in lacerating a cardiac valve leaflet.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to place a stent, or perform balloon angioplasty, in one of the coronary arteries. In some instances, it may be beneficial to slice or lacerate one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking cardiac artery ostiums when displayed to the side when a second artificial heart valve is implanted in place of the first artificial heart valve. The artificial valve leaflets forming part of the artificial heart valve, which may for example be made from bovine tissue, or may be polymeric. In some instances, artificial valve leaflets may be made of polymers such as Dacron or Gore-Tex. As discussed here, reference to a valve leaflet may refer to either a native valve leaflet or an artificial valve leaflet.

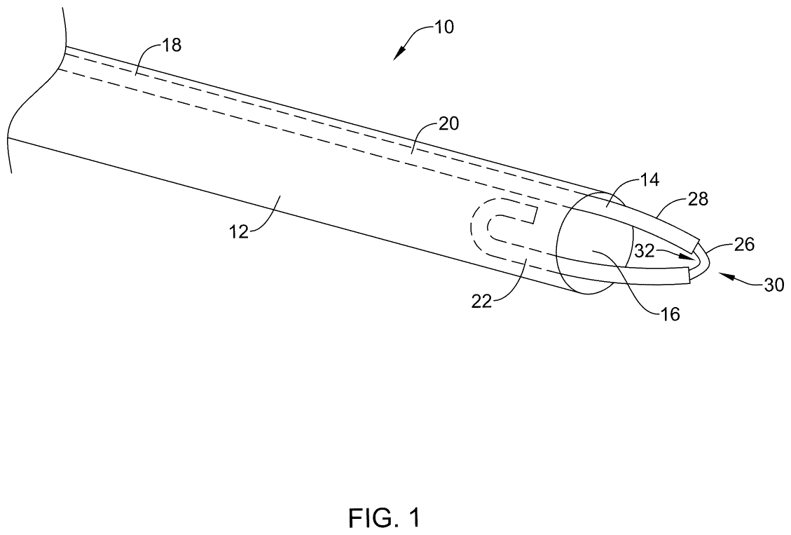

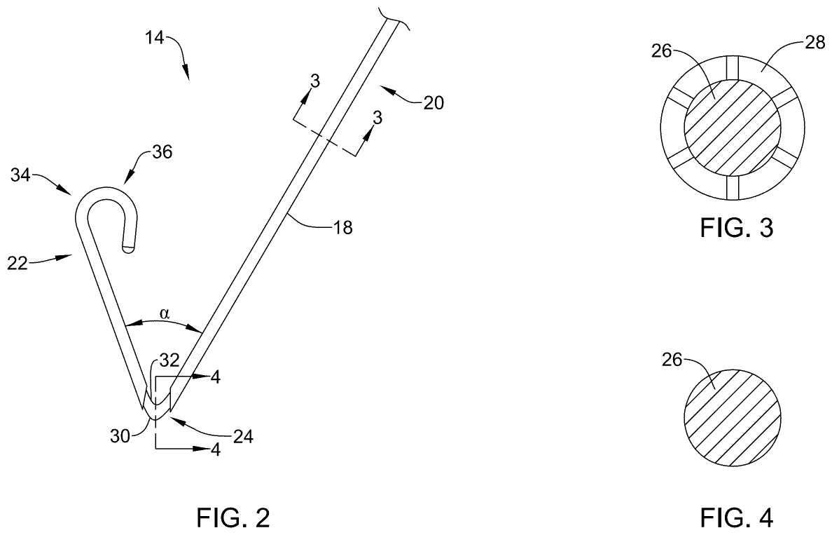

FIG. 1 is a schematic view of an illustrative medical assembly 10 that may be used for puncturing and subsequently lacerating one of the heart valve leaflets, which may be either native heart valve leaflets, or artificial heart valve leaflets. While the medical assembly 10 may be adapted to puncture and subsequently lacerate one heart valve leaflet at a time, it will be appreciated that multiple instances of the medical assembly 10 may be used to puncture and lacerate more than one heart valve leaflet. In some instances, multiple instances of the medical assembly 10 may be used to perform more than one puncture and laceration in a single heart valve leaflet, albeit one puncture and one laceration per medical assembly 10. The medical assembly 10 includes a guide catheter 12 and an electrocautery member 14, which is also shown in FIG. 2. The electrocautery member 14 may be adapted to be slidingly disposed within a lumen 16 extending through the guide catheter 12 in order to puncture and lacerate a native cardiac valve leaflet. In some instances, the electrocautery member 14 may be considered as being a single shaft electrocautery device, where the single shaft electrocautery device is used to initially puncture the cardiac valve leaflet and is subsequently to slice or lacerate the cardiac valve leaflet.

In some instances, the guide catheter 12 and the electrocautery member 14 may be used together to allow a cardiac valve leaflet to be first punctured and then lacerated without actually excising any part of the cardiac valve leaflet. For example, the medical assembly 10 may be advanced to a position proximate a cardiac valve leaflet in a configuration shown in FIG. 1, with the electrocautery member 14 largely disposed within the lumen 16. This may be considered as being a puncture position. As will be described, after the electrocautery member 14 is used to form a puncture in the cardiac valve leaflet, the electrocautery member 14 may be advanced out of the lumen 16 and through the puncture to reach a lacerate position in which the electrocautery member 14 is able to slice or lacerate from the puncture to an outer edge of the cardiac valve leaflet, thereby slicing or lacerating the cardiac valve leaflet. The electrocautery member 14 may utilize radiofrequency (RF) energy in forming a puncture within the cardiac valve leaflet. The electrocautery member 14 may utilize RF energy in forming the slit or laceration extending from the puncture to an outer edge of the cardiac valve leaflet.

The electrocautery member 14 includes a single shaft 18 having a proximal shaft segment 20, a distal shaft segment 22 and a hinge segment 24 (shown in FIG. 2) that is disposed between the proximal shaft segment 20 and the distal shaft segment 22. FIG. 2 is a schematic view of an illustrative electrocautery member forming part of the illustrative medical assembly of FIG. 1. In some cases, as shown, the hinge segment 24 represents a bend in the shaft 18. In some cases, the hinge segment 24 may be longer than just being a bend in the shaft 18. The single shaft 18 includes a conductive core 26 and an insulative polymeric sheath 28 that extends over the proximal shaft segment 20 and the distal shaft segment 22. In some instances, the single shaft 18 may include echogenic features that enhance visibility of the single shaft 18 when viewed under ultrasound. This may include etching or grooves formed within either the conductive core 26, the insulative polymeric sheath 28, or both. In some instances, additional parts such as a coil may be added under the insulative polymeric sheath 28.

The insulative polymeric sheath 28 does not extend over all of the hinge segment 24. In some instances, the insulative polymeric sheath 28 does not extend over any of the hinge segment 24, leaving the hinge segment 24 uncovered and thus able to be electrically active when RF energy is applied to the conductive core 26. FIG. 3 is a cross-sectional view taken along the line 3-3 of FIG. 2, showing the conductive core 26 within the insulative polymeric sheath 28. FIG. 4 is a cross-sectional view taken along the line 4-4 of FIG. 2, showing how the conductive core 26 is not covered by the insulative polymeric sheath 28 within the hinge segment 24.

In some instances, the hinge segment 24 may be considered as including a leading edge 30 that is adapted to form a puncture within a cardiac valve leaflet and a trailing edge 32 that is adapted to lacerate the cardiac valve leaflet. In some instances, the leading edge 30 and the trailing edge 32 are each exposed portions of the conductive core 26, and hence the leading edge 30 and the trailing edge 32 may be electrically coupled. In some instances, while not shown, the leading edge 30 and the trailing edge 32 may be electrically isolated from each other, and thus the electrocautery member 14 may include two distinct conductive members, a first conductive member electrically coupled with the leading edge 30 and a second conductive member electrically coupled with the trailing edge 32 yet electrically isolated from the first conductive member.

In some instances, the proximal shaft segment 20 and the distal shaft segment 22 may form an angle α (alpha) therebetween. In some instances, the angle α (alpha) may be considered as being an acute angle, meaning that the angle α (alpha) is less than 90 degrees. In some instances, the angle α (alpha) may change depending on whether the electrocautery member 14 is in its puncture position (as seen for example in FIG. 1) or the electrocautery member 14 is in its lacerate position (as seen for example in FIG. 2). The angle α (alpha) may also vary, depending on the inner diameter of the guide catheter 12, for example. In some instances, the electrocautery member 14 may be considered as being biased to the lacerate position. In some instances, the lacerate position may represent a remembered position. The conductive core 26 may be formed of a shape memory metal such as nitinol. As an example, the angle α (alpha) may vary from about 20 to about 30 degrees when in the puncture position (with the distal shaft segment 22 extending proximally into the lumen 16 and the angle α (alpha) may vary from about 40 to about 50 degrees when in the lacerate position. This is just an example, and other relative geometries are contemplated.

In some instances, the electrocautery member 14 may be considered as being adapted to be disposed relative to the guide catheter 12 with the distal shaft segment 22 extending proximally within the lumen 16 in order to position the leading edge 30 of the hinge segment 24 to form a puncture within the cardiac valve leaflet. In some instances, the electrocautery member 14 may be adapted to be disposed relative to the guide catheter 12 with the distal shaft segment 22 extending distally of the lumen 16 in order to position the trailing edge 32 of the hinge segment 24 to lacerate the cardiac valve leaflet.

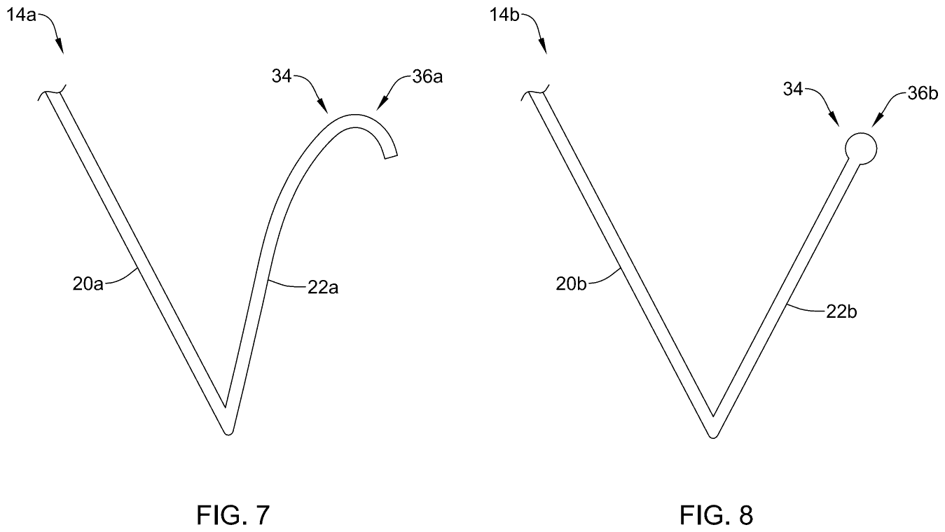

In some instances, the distal shaft segment 22 may include a distal region 34 in which the distal shaft segment 22 includes an atraumatic tip 36. In some instances, as shown in FIG. 2, the atraumatic tip 36 may be formed by the distal region 34 of the distal shaft segment 22 doubling over on itself. As shown, the distal region 34 of the distal shaft segment 22 bends in a direction towards the proximal shaft segment 20. It will be appreciated that these are two-dimensional representations of what is actually a three-dimensional structure. In some instances, the atraumatic tip 36 may actually curve in a direction into a plane of the paper, or in a direction out of (towards the viewer) out of the plane of the paper. In some instances, the distal shaft segment 22 and the atraumatic tip 36 may lie within the plane of the paper. In some instances, the distal shaft segment 22 may terminate close to the hinge segment 24. Other configurations are also possible, as will be discussed for example with respect to FIGS. 7 and 8.

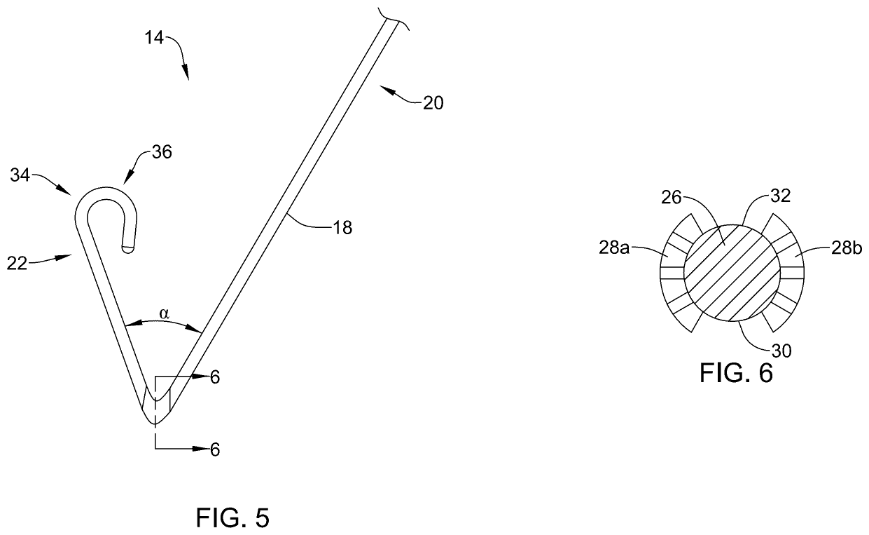

In some instances, at least part of the hinge segment 24 may be covered by the insulative polymeric sheath 28. FIG. 5 is a schematic view of the electrocautery member 14 in which the insulative polymeric sheath 28 covers what might be considered (in the illustrated orientation) a front and a back of the hinge segment 24, but does not cover the leading edge 30 or the trailing edge 32. This is illustrated in FIG. 6, which is a cross-sectional view taken along the line 6-6 of FIG. 5. As can be seen, the leading edge 30 is not covered by the insulative polymeric sheath 30 and the trailing edge 32 is not covered by the insulative polymeric sheath 30. The insulative polymeric sheath 30 includes a first segment 28a covering a first side of the conductive core 26 and a second segment 28b covering a second side of the conductive core 26.

In some instances, the first segment 28a and the second segment 28b may extend beyond the conductive core 26. As an example, the first segment 28a and the second segment 28b may form insulative flaps that can provide more or less electrode coverage, depending on position. The insulative flaps can extend in a first direction or a second direction, depending on a direction of motion of the electrocautery member 14. For example, the insulative flaps may cover more of the trailing edge 32 when the electrocautery member 14 is in the puncture position. The insulative flaps may cover more of the leading edge 30 when the electrocautery member 14 is in the lacerate position, for example. In some instances, this will help in concentrating the current density at the desired RF surface for puncturing (in the puncture position) or for cutting (in the lacerate position).

As noted, FIG. 2 shows the distal region 34 of the distal shaft segment 22 as including an atraumatic tip 36 in which the distal region 34 of the distal shaft segment 22 curves or bends towards the proximal shaft segment 20. FIG. 7 is a schematic view of an illustrative electrocautery member 14a having a proximal shaft segment 20a and a distal shaft segment 22a. The distal shaft segment 22a includes a distal region 34. An atraumatic tip 36a is formed by the distal region 34 of the distal shaft segment 22a curving or bending in a direction away from the proximal shaft segment 20a. FIG. 8 is a schematic view of an illustrative electrocautery member 14b having a proximal shaft segment 20b and a distal shaft segment 22b. The distal shaft segment 22b includes a distal region 34. In this example, an atraumatic tip 36b take the form of a bulbous element. The bulbous element may be a widened portion of the distal region 34 of the distal shaft segment 22b. In some instances, the bulbous element may be welded or soldered to the distal shaft segment 22b. The atraumatic tip 36b may be an expandable basket, for example. In some instances, the atraumatic tip 36b may be a balloon. Other curved shapes are also contemplated. It will be appreciated that these are two-dimensional representations of what is actually a three-dimensional structure. In some instances, the atraumatic tip 36a shown in FIG. 7 and the atraumatic tip 36b shown in FIG. 8 may actually curve in a direction into a plane of the paper, or in a direction out of (towards the viewer) out of the plane of the paper. In some instances, the atraumatic tip 36 may actually curved within the plane of the paper.

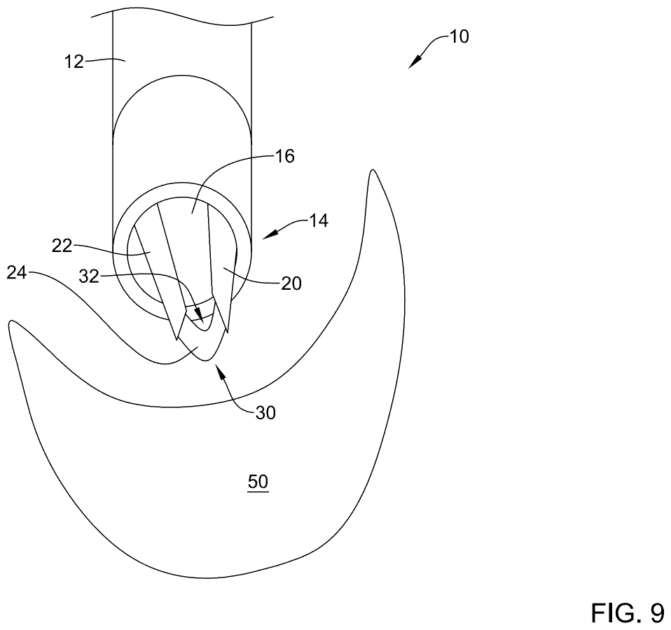

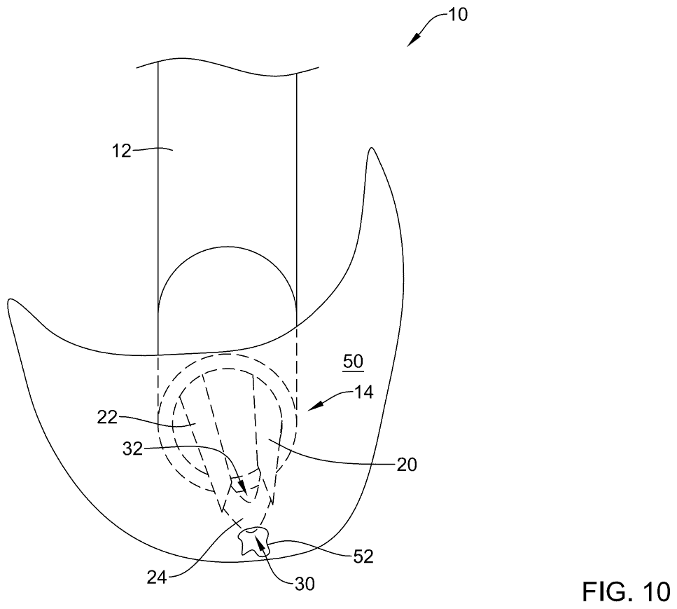

FIGS. 9 through 14 illustrate use of the medical assembly 10 in puncturing and lacerating a native cardiac valve leaflet in order to avoid any possible problems with one or more of the native cardiac valve leaflets substantially (defined as more than fifty percent blockage) blocking or even completely blocking an ostium of one or more coronary arteries when the native cardiac valve leaflets are pushed to the side when an artificial heart valve is implanted. It will be appreciated that an artificial cardiac valve leaflet may be punctured and lacerated, without removal, in a similar fashion. In FIG. 9, the medical assembly 10 is positioned proximate a native cardiac valve leaflet 50. The electrocautery member 14 is in the puncture position, with the distal shaft segment 22 extending proximally within the lumen 16, and the hinge segment 24 being the distal-most portion of the electrocautery member 14. The leading edge 30 of the hinge segment 24 may be seen as approaching the native cardiac valve leaflet 50. In the puncture position, the distal shaft segment 22 remains held in position by remaining at least partially within the guide catheter 12.

In FIG. 10, the electrocautery member 14 has been advanced distally to a point where the leading edge 30 of the hinge segment 24 contacts the native cardiac valve leaflet 50. RF energy is applied to form a puncture 52. In some instances, the hinge segment 24 may instead be adapted to mechanically form the puncture 52. The distal shaft segment 22 remains held in the puncture position by virtue of part of the distal shaft segment 22 remaining within the guide catheter 12.

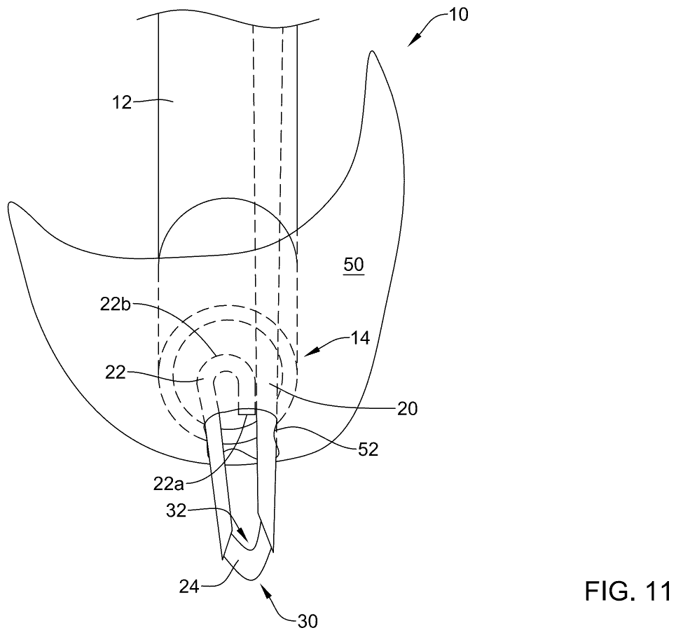

In FIG. 11, the electrocautery member 14 has been advanced distally through the puncture 52 to a point where a distal end 22a of the distal shaft segment 22 has advanced through the puncture 52 while a curved portion 22b, which forms part of the atraumatic tip of the distal shaft segment 22, remains within the guide catheter 12. This helps to hold the distal shaft segment 22 in the puncture position, in which the distal shaft segment 22 is held more tightly to the proximal shaft segment 20. In some instances, rather than advancing the electrocautery member 14 distally into and through the puncture 52, the guide catheter 12 itself may be advanced into the puncture 52 with the electrocautery member 14 extending distally out of the guide catheter 12.

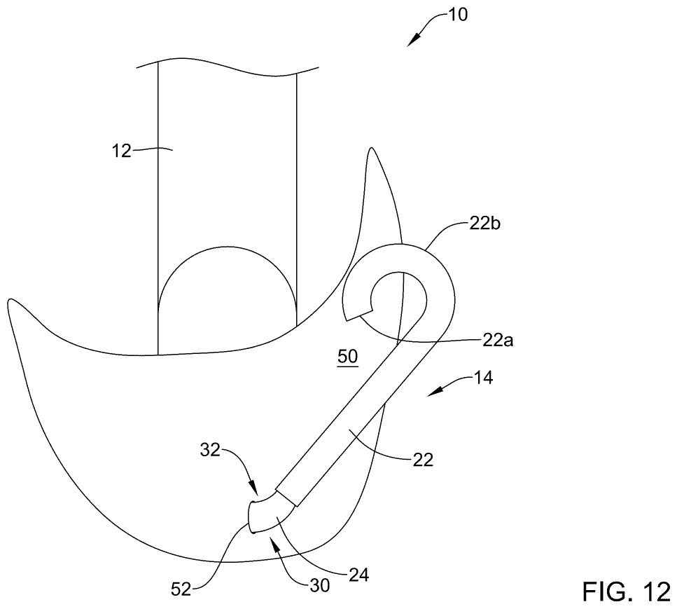

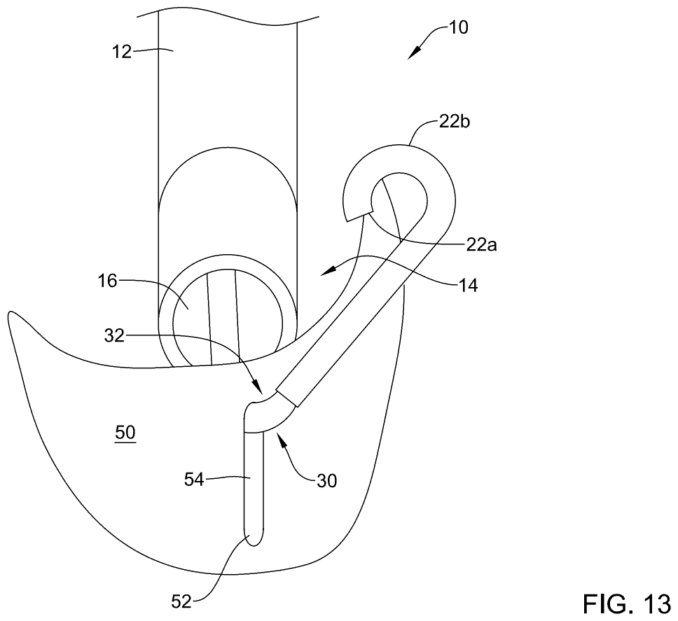

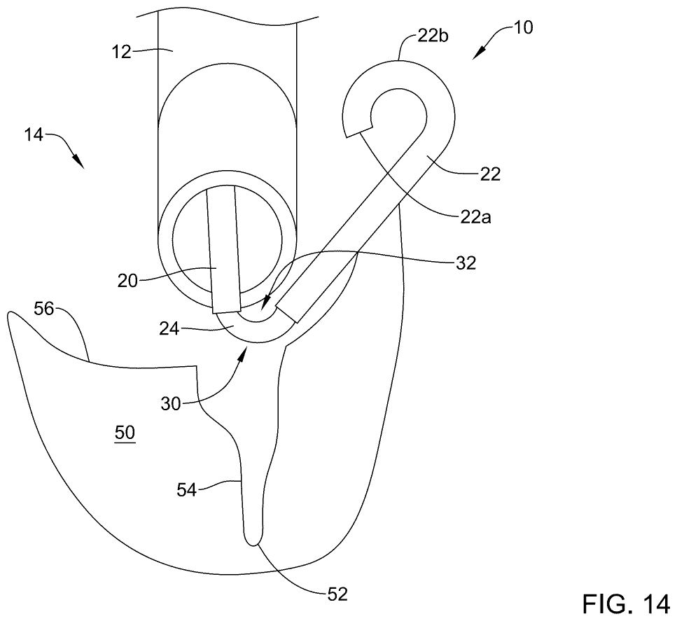

In FIG. 12, the electrocautery member 14 has been pushed through the puncture 52 to the point at which the distal shaft segment 22 is released from the lumen 16, including releasing the curved portion 22b of the distal shaft segment 22, and now extends beyond the puncture 52. In FIG. 13, the electrocautery member 14 is being withdrawn proximally, such that the trailing edge 32 of the hinge segment 24 contacts the native cardiac valve leaflet 50 and forms a slit 54 that extends in an upward direction (in the illustrated orientation) through the native cardiac valve leaflet 50 as RF energy is applied and the electrocautery member 14 is moved proximally. In some instances, the hinge segment 24 may instead be adapted to mechanically form the slit 54. As can be seen in FIG. 14, continued withdrawal of the electrocautery member 14 while applying RF energy causes the trailing edge 32 of the hinge segment 24 to continue to enlarge the slit 54 until reaching an edge 56 of the native cardiac valve leaflet 50. The native cardiac valve leaflet 50 is now less likely to impede or otherwise block an ostium of a coronary artery when an artificial heart valve is subsequently implanted. Further proximal withdrawal of the electrocautery member 14 relative to the guide catheter 12 (or distal advancement of the guide catheter 12 relative to the electrocautery member 14) will cause the distal shaft segment 22 of the electrocautery member 14 to be pulled into the lumen 16 for withdrawal of the medical assembly 10.

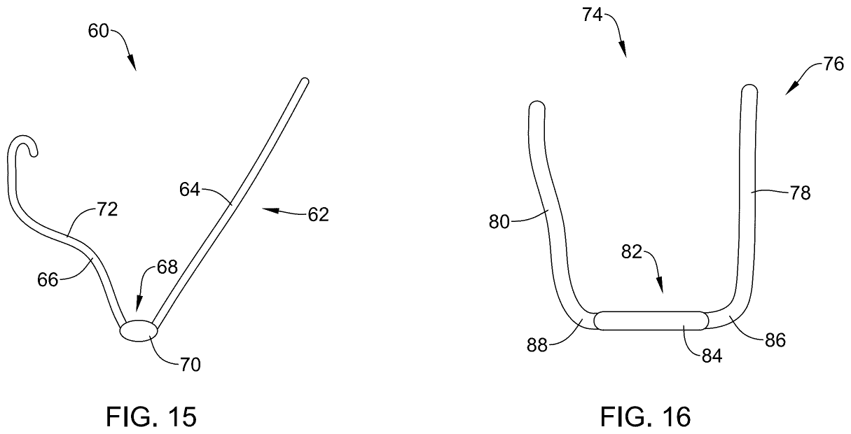

FIG. 15 is a schematic view of an illustrative electrocautery member 60 that may be used in place of the electrocautery member 14 as part of the medical assembly 10 shown in FIG. 1. In some instances, the guide catheter 12 and the electrocautery member 60 may be used together to allow a cardiac valve leaflet to be first punctured and then lacerated without actually excising any part of the cardiac valve leaflet. As will be described, after the electrocautery member 60 is used to form a puncture in the cardiac valve leaflet, the electrocautery member 60 may be advanced out of the lumen 16 and through the puncture to reach a lacerate position in which the electrocautery member 60 is able to slice or lacerate from the puncture to an outer edge of the cardiac valve leaflet, thereby slicing or lacerating the cardiac valve leaflet. The electrocautery member 60 may utilize RF energy in forming a puncture within the cardiac valve leaflet. The electrocautery member 60 may utilize RF energy in forming the slit or laceration extending from the puncture to an outer edge of the cardiac valve leaflet.

The electrocautery member 60 includes a shaft 62 having a proximal shaft segment 64, a distal shaft segment 66, and a hinge segment 68 that is disposed between the proximal shaft segment 64 and the distal shaft segment 66. In some cases, the shaft 62 includes a conductive core that is covered with an insulative polymeric sheath, much like the conductive core 26 and the insulative polymeric sheath 28. The hinge segment 68 may include an electrode 70 that may be used to first form a puncture within the tissue of the cardiac valve leaflet and then used to lacerate the cardiac valve leaflet. In some cases, the electrode 70 may represent a portion of the conductive core from which the insulative polymeric sheath has been removed.

In some cases, the distal shaft segment 66 may remain within the guide catheter 12 when the electrode 70 is being used to puncture the cardiac valve leaflet, and the electrocautery member 60 is then pushed through the opening that is punctured into the cardiac valve leaflet. As the distal shaft segment 66 and a distal portion of the proximal shaft segment 64 are pushed through the opening, the electrocautery member 60 reverts to its remembered profile (as shown) in which the distal shaft segment 66 extends from the hinge segment 68 at an acute angle relative to the proximal shaft segment 64. In some cases, the distal shaft segment 66 may include an additional curve 72 that is formed into the distal shaft segment 66. In some cases, the additional curve 72 may help the electrocautery member 60 return to its remembered shape (as shown) after the electrode 70 has been used to form the initial puncture within the cardiac valve leaflet.

FIG. 16 is a schematic view of an illustrative electrocautery member 74 that may be used in place of the electrocautery member 14 as part of the medical assembly 10 shown in FIG. 1. In some instances, the guide catheter 12 and the electrocautery member 74 may be used together to allow a cardiac valve leaflet to be first punctured and then lacerated without actually excising any part of the cardiac valve leaflet. As will be described, after the electrocautery member 74 is used to form a puncture in the cardiac valve leaflet, the electrocautery member 74 may be advanced out of the lumen 16 and through the puncture to reach a lacerate position in which the electrocautery member 74 is able to slice or lacerate from the puncture to an outer edge of the cardiac valve leaflet, thereby slicing or lacerating the cardiac valve leaflet. The electrocautery member 74 may utilize RF energy in forming a puncture within the cardiac valve leaflet. The electrocautery member 74 may utilize RF energy in forming the slit or laceration extending from the puncture to an outer edge of the cardiac valve leaflet.

The electrocautery member 74 includes a shaft 76 having a proximal shaft segment 78, a distal shaft segment 80, and an intermediate segment 82 that is disposed between the proximal shaft segment 78 and the distal shaft segment 80. In some cases, the shaft 76 includes a conductive core that is covered with an insulative polymeric sheath, much like the conductive core 26 and the insulative polymeric sheath 28. The intermediate segment 82 may include an electrode 84. In some cases, the electrode 84 may represent a portion of the conductive core from which the insulative polymeric sheath has been removed. In some cases, the proximal shaft segment 78 includes a bend 86 that is adjacent to and proximal of the intermediate segment 82. In some cases, the distal shaft segment 80 includes a bend 88 that is adjacent to and distal of the intermediate segment 82. In some cases, the bend 86 and the bend 88 may help the electrocautery member 74 to regain its remembered profile, as shown.

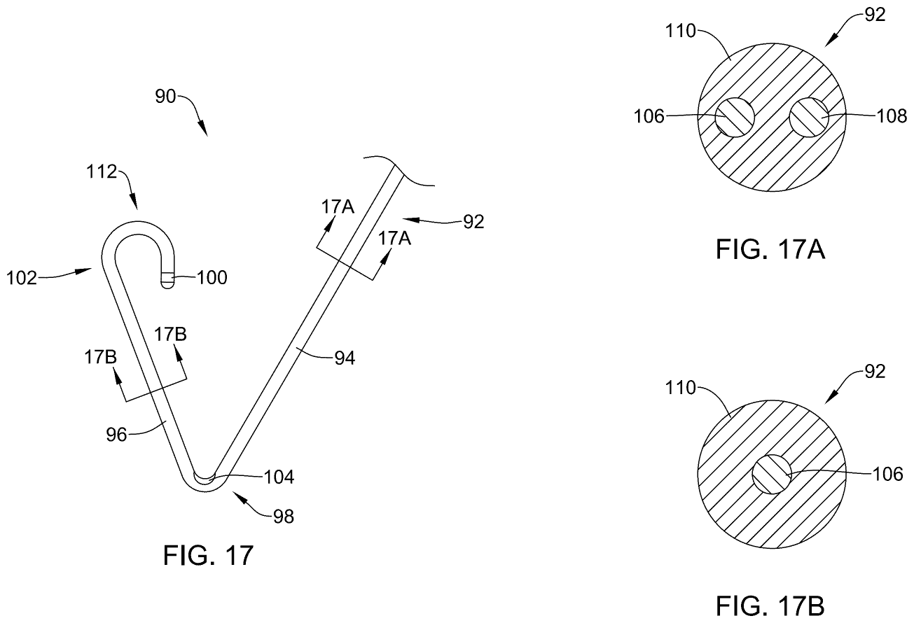

FIG. 17 is a schematic view of an illustrative electrocautery member 90. FIGS. 17A and 17B are cross-sectional views, taken along the lines 17A-17A and 17B-17B of FIG. 17, respectively. The electrocautery member 90 may be used in place of the electrocautery member 14 as part of the medical assembly 10 shown in FIG. 1. In some instances, the guide catheter 12 and the electrocautery member 90 may be used together to allow a cardiac valve leaflet to be first punctured and then lacerated without actually excising any part of the cardiac valve leaflet. As will be described, after the electrocautery member 90 is used to form a puncture in the cardiac valve leaflet, the electrocautery member 90 may be advanced out of the lumen 16 and through the puncture to reach a lacerate position in which the electrocautery member 90 is able to slice or lacerate from the puncture to an outer edge of the cardiac valve leaflet, thereby slicing or lacerating the cardiac valve leaflet. The electrocautery member 90 may utilize RF energy in forming a puncture within the cardiac valve leaflet. The electrocautery member 90 may utilize RF energy in forming the slit or laceration extending from the puncture to an outer edge of the cardiac valve leaflet.

The electrocautery member 90 includes a shaft 92 having a proximal shaft segment 94, a distal shaft segment 96, and a hinge segment 98 that is disposed between the proximal shaft segment 94 and the distal shaft segment 96. A puncture electrode 100 is disposed within a distal end region 102 of the distal shaft segment 96. A lacerating electrode 104 is disposed at or near the hinge segment 98. In some cases, the puncture electrode 100 and the lacerating electrode 104 are electrically separated from each other. This allows RF energy to be more concentrated when applied sequentially to the puncture electrode 100 and the lacerating electrode 104, rather than applying RF energy to both the puncture electrode 10 and the lacerating electrode 104 simultaneously. In some cases, the shaft 92 may include a first conductive member 106 that is electrically coupled with the puncture electrode 100 and a second conductive member 108 that is electrically coupled with the lacerating electrode 104. The first conductive member 106 and the second conductive member 108 are electrically isolated from each other via an electrically insulating material 110. In some cases, the first conductive member 106 and the second conductive member 108 may each be formed of nitinol.

In some cases, the puncture electrode 100 may be a part of the first conductive member 106 that is exposed by a portion of the electrically insulating material 110 being removed. In some cases, the puncture electrode 110 may include or be formed by a terminus of the first conductive member 106. In some cases, the lacerating electrode 104 may be a part of the second conductive member 108 that is exposed by a portion of the electrically insulating material 110 being removed. In some cases, the lacerating electrode 104 may include or be formed by a terminus of the second conductive member 108. It will be appreciated that the cross-sectional views shown in FIGS. 17A and 17B merely represent one possible arrangement of how the shaft 92 accommodates the first conductive member 106 and the second conductive member 108. In some cases, the distal shaft segment 96 may have a smaller outer diameter than the proximal shaft segment 94 as a result of the proximal shaft segment 94 accommodating both the first conductive member 106 and the second conductive member 108 while the distal shaft segment 96 only accommodates the first conductive member 106 and does not include the second conductive member 108 extending therethrough. In some cases, as shown, the distal shaft segment 96 may include an atraumatic tip 112.

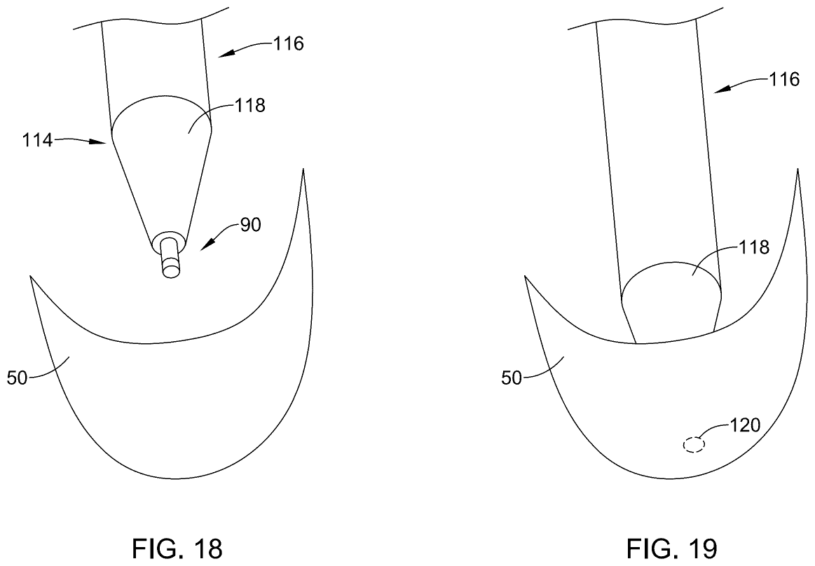

FIGS. 18 through 21 illustrate use of an illustrative medical assembly 114 in puncturing and puncturing and lacerating a native cardiac valve leaflet in order to avoid any possible problems with one or more of the native cardiac valve leaflets substantially (defined as more than fifty percent blockage) blocking or even completely blocking an ostium of one or more coronary arteries when the native cardiac valve leaflets are pushed to the side when an artificial heart valve is implanted. The medical assembly 114 includes the electrocautery member 90 shown disposed within a guide catheter 116. The guide catheter 116 may be similar to the guide catheter 12. In some cases, as shown, the guide catheter 116 may include a tapered distal region 118, but this is not required in all cases.

It will be appreciated that an artificial cardiac valve leaflet may be punctured and lacerated, without removal, in a similar fashion. In FIG. 18, the medical assembly 114 is positioned proximate the native cardiac valve leaflet 50 with the puncture electrode 100 extending out of the tapered distal region 118 of the guide catheter 116. The puncture electrode 100 is positioned to form a puncture within the native cardiac valve leaflet 50. In FIG. 19, the medical assembly 114 has been advanced distally to a position in which the puncture electrode 100 is adjacent the native cardiac valve leaflet 50, and RF energy is being applied to the puncture electrode 100. As a result, a puncture 120 (shown in phantom line) is beginning to form within the native cardiac valve leaflet 50.

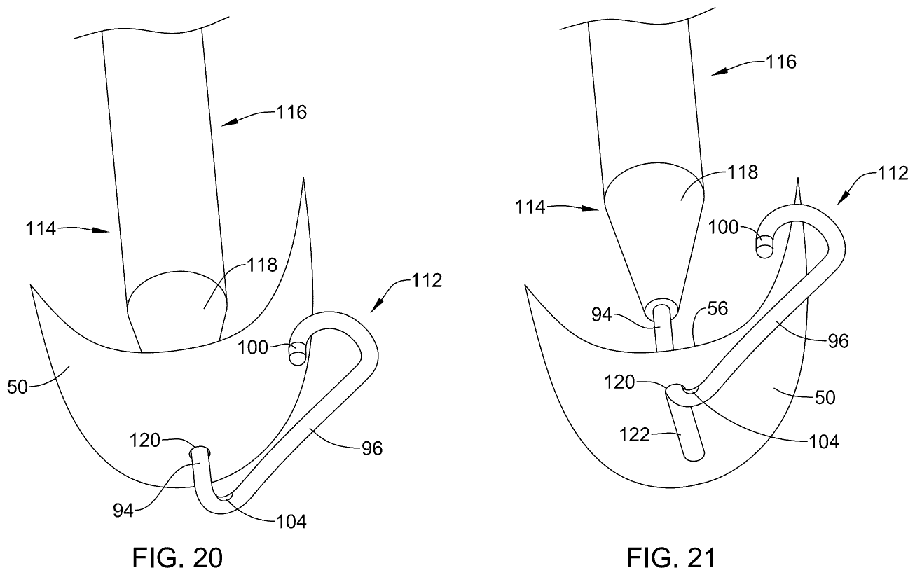

In FIG. 20, the electrocautery member 90 has been advanced through the puncture 120 such that the distal shaft segment 96 has passed through the puncture 120 and has regained its remembered profile (as shown). The lacerating electrode 104 is positioned such that withdrawing the electrocautery member 90 while applying RF energy to the lacerating electrode 104 causes the lacerating electrode 104 to lacerate the native cardiac valve leaflet 50, thereby creating a laceration 122. While not shown, continuing to apply RF energy to the lacerating electrode 104 while continuing to withdraw the electrocautery member 90 causes the lacerating electrode 104 to continue to enlarge the laceration 122 until the lacerating 122 reaches the edge 56 the of native cardiac valve leaflet 50. Further proximal withdrawal of the electrocautery member 90 relative to the guide catheter 116 (or distal advancement of the guide catheter 116 relative to the electrocautery member 90) will cause the distal shaft segment 96 of the electrocautery member 90 to be pulled into an interior of the guide catheter 116 for withdrawal of the medical assembly 114.

The materials that can be used for the various components of the medical assembly 10 and the various elements thereof disclosed herein may include those commonly associated with medical devices. In some instances, the medical assembly 10, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylenc/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/cthers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrenc) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical assembly 10, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical assembly 10 and/or other elements disclosed herein. For example, the medical assembly 10, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical assembly 10 and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical assembly, comprising:
a guide catheter including a distal end and a lumen extending proximally from the distal end; and
an electrocautery member adapted to be slidingly disposed within the lumen in order to puncture and lacerate a cardiac valve leaflet, the electrocautery member comprising:
a shaft including:
a proximal shaft segment;
a distal shaft segment including a distal end region; and
a hinge segment disposed between the proximal shaft segment and the distal shaft segment;
a puncture electrode disposed relative to the shaft; and
a lacerating electrode disposed relative to the shaft;
wherein the electrocautery member is adapted to lacerate the cardiac valve leaflet without excising part of the cardiac valve leaflet.

2. The medical assembly of claim 1, wherein the puncture electrode is disposed within the distal end region of the distal shaft segment.

3. The medical assembly of claim 1, wherein the lacerating electrode comprises an electrically exposed portion of the hinge segment.

4. The medical assembly of claim 1, wherein the puncture electrode comprises a leading edge of the hinge segment.

5. The medical assembly of claim 1, wherein the lacerating electrode comprises a trailing edge of the hinge segment.

6. A medical assembly, comprising:
a guide catheter including a distal end and a lumen extending proximally from the distal end; and
an electrocautery member adapted to be slidingly disposed within the lumen in order to puncture and lacerate a cardiac valve leaflet, the electrocautery member having a single shaft comprising:
a proximal shaft segment;
a distal shaft segment; and
a hinge segment disposed between the proximal shaft segment and the distal shaft segment, the hinge segment including:
a leading edge adapted to form a puncture within the cardiac valve leaflet; and
a trailing edge adapted to lacerate the cardiac valve leaflet;
wherein the electrocautery member is adapted to lacerate the cardiac valve leaflet without excising part of the cardiac valve leaflet.

7. The medical assembly of claim 6, wherein the single shaft comprises:
a conductive core extending through the proximal shaft segment, the hinge segment and the distal shaft segment; and
an insulative polymeric sheath extending over the conductive core, the insulative polymeric sheath electrically insulating the proximal shaft segment and the distal shaft segment;
wherein the leading edge of the hinge segment is not covered by the insulative polymer sheath; and
wherein the trailing edge of the hinge segment is not covered by the insulative polymer sheath.

8. The medical assembly of claim 7, wherein the conductive core comprises nitinol.

9. The medical assembly of claim 6, wherein the electrocautery member is adapted to be disposed relative to the guide catheter with the distal shaft segment extending proximally within the lumen in order to position the leading edge of the hinge segment to form the puncture within the cardiac valve leaflet.

10. The medical assembly of claim 9, wherein the electrocautery member is adapted to be disposed relative to the guide catheter with the distal shaft segment extending distally of the lumen in order to position the trailing edge of the hinge segment to lacerate the cardiac valve leaflet.

11. The medical assembly of claim 6, wherein the proximal shaft segment and the distal shaft segment form an acute angle at the hinge segment.

12. The medical assembly of claim 6, wherein the distal shaft segment includes an atraumatic tip formed at a distal region of the distal shaft segment.

13. A medical assembly, comprising:
a guide catheter including a distal end and a lumen extending proximally from the distal end; and
an electrocautery member adapted to be slidingly disposed within the lumen and to puncture and lacerate a cardiac valve leaflet without excising the cardiac valve leaflet, the electrocautery member including:
a shaft including:
a proximal shaft segment; and
a distal shaft segment including a distal end region;
a puncture electrode disposed within the distal end region; and
a lacerating electrode disposed between the proximal shaft segment and the distal shaft segment.

14. The medical assembly of claim 13, wherein the shaft comprises:
a first conductive member electrically coupled with the puncture electrode; and
a second conductive member electrically coupled with the lacerating electrode.

15. The medical assembly of claim 14, wherein the first conductive member and the second conductive member are electrically isolated from each other via an electrically insulating material encasing the first conductive member and the second conductive member.

16. The medical assembly of claim 15, wherein the puncture electrode comprises a portion of the first conductive member exposed by a portion of the electrically insulating material being removed.

17. The medical assembly of claim 15, wherein the puncture electrode comprises a terminus of the first conductive member.

18. The medical assembly of claim 15, wherein the lacerating electrode comprises a portion of the second conductive member exposed by a portion of the electrically insulating material being removed.

19. The medical assembly of claim 15, wherein the lacerating electrode comprises a terminus of the second conductive member.

20. The medical assembly of claim 14, wherein the first conductive member and the second conductive member each comprise nitinol.
